(19) United States
(12) Patent Application Publication
(10) Pub. No.: US 2024/0350196 A1
(43) Pub. Date: Oct. 24, 2024

(54) Method of Preventing Sloughing or Incomplete Vaporization of Tissues During a Laser Ablation Procedure

(71) Applicant: OPTICAL INTEGRITY, INC., PANAMA CITY BEACH, FL (US)

(72) Inventor: JOE D. BROWN, PANAMA CITY BEACH,, FL (US)

(73) Assignee: OPTICAL INTEGRITY, INC., (omitted)

(21) Appl. No.: 18/642,086

(22) Filed: Apr. 22, 2024

Related U.S. Application Data
(60) Provisional application No. 63/497,451, filed on Apr. 21, 2023.

(51) Int. Cl. A61B 18/24; A61B 18/20

(52) U.S. Cl. CPC A61B 18/24; A61B 2018/205547

(57) ABSTRACT

A method for preventing undesired tissue coagulation or sloughing during a laser ablation or vaporization procedure involves setting a predetermined usage limit based on objective criteria indicative of wear and/or debris accumulation on a catheter or sleeve through which a surgical laser is fired, and replacing or re-positioning the catheter or sleeve when the predetermined usage limit is exceeded.

### BACKGROUND OF THE INVENTION

### 1. Field of the Invention

The invention relates to field of laser surgery, and in particular to systems and devices for delivering laser radiation to vaporize or ablate a targeted tissue. The surgical procedures may include, but are not limited to, varicose vein ablation and benign prostate hyperplasia (BPH) treatment.

More particularly, the invention involves a method that addresses a heretofore unappreciated problem caused by wear or accumulation of debris on a sleeve or catheter through which a laser beam is fired. While it is generally recognized that wear or debris accumulation at the site where a laser beam exits the sleeve will result in a loss of beam intensity and increased treatment times, the inventors have discovered that the loss of beam intensity may also result in coagulation or sloughing, in effect “cooking,” the targeted tissue, which can limit ablation and cause loss of tissue function, exacerbating the problem that the treatment procedure was intended to solve.

The method of the invention sets a limit to the number of times that the sleeve or catheter may be used before requiring replacement of the sleeve or catheter, or re-positioning so that the laser beam is fired through a portion of the sleeve or catheter that has not yet been degraded or subjected to debris accumulation. The sleeve or catheter may include transparent sections, windows, or apertures through which the laser is fired at an angle relative to the axis of the laser delivery fiber.

### 2. Description of Related Art

For side-firing laser fibers, protection of the fiber has conventionally involved use of a transparent cap through which the laser is fired, and that is permanently affixed to the optical fiber to form a laser delivery fiber assembly. The side-firing fiber assembly may be inserted into or combined with an external tube or sleeve that enables flow of irrigation fluid past the fiber tip, in order to cool and flush debris away from the fiber. A disadvantage of this arrangement, known as the Moxy™ system, is that the permanently affixed transparent cap will accumulate debris, causing degradation of the cap and increasing attenuation of the laser as it is fired through the cap. In order to prevent excessive debris accumulation, frequent cleaning of the cap and/or frequent replacement of the entire fiber/cap arrangement is necessary, which results in high cost and extended treatment times.

An alternative to the Moxy™ system is the system disclosed in U.S. Pat. No. 6,802,838 (the Trimedyne™ system), which replaces the Moxy™ cap and sleeve with a catheter that is crimped to the fiber or secured thereto by an insert, and that includes a window through which the laser is fired, as well as multiple ports for fluid input and debris removal. Because the catheter is fixed to the fiber, the Trimedyne™ system suffers at least the same cost and treatment-time disadvantages as the Moxy™ system. In addition, the Trimedyne™ system as the additional disadvantage of providing only a single window for emission of the radiation, with the result that degradation caused by the laser is concentrated in the area of the window.

Copending U.S. patent application Ser. No. 15/957,085, filed Apr. 19, 2018, now U.S. Pat. No. 11,109,912, and also incorporated by reference herein, discloses further variations of conventional side-firing laser systems such the Moxy™ and Trimedyne™ catheters or sleeves. These further variations include designs in which the outer sleeve is in the form of a catheter that, unlike the catheter of the Trimedyne™ system, is modified so that it can be easily removed and replaced without having to also dispose of the fiber, and which includes a firing window that extends circumferentially around the catheter to enable the fiber to be rotated within the catheter in order to limit damage caused by firing of the laser to any one area of the catheter.

The sleeves or catheters disclosed in the copending applications include embodiments in which the sleeve or catheter is separable from the fiber for disposal and replacement, allowing the sleeve or catheter to be replaced without having to replace the more expensive fiber. Not only does this provide a simple and inexpensive way to extend fiber life and avoid the need for time-consuming fiber replacement during a surgical procedure, but it provides a degree of modularity, allowing interchangeability of catheter tips. Nevertheless, even though replacement of a catheter does not require a great deal of time, any interruption in a surgical procedure will increase the duration of the procedure and risk to the patient, and therefore sleeves and catheters have been proposed that can be re-positioned relative to the tip of the fiber from which the laser beam is emitted, allowing a degraded portion of the sleeve or catheter to be moved away from the fiber tip, without having to replace the sleeve or catheter itself. For example, copending U.S. patent application Ser. No. 17/135,225, filed Dec. 28, 2020, and published as U.S. Patent Publication No. 2021/0196379, discloses an improvement over the removable catheter, in which multiple ports are provided. When wear occurs around one opening, the multiple port configuration allows continued use of the catheter, without the need for immediate replacement, by simply moving the fiber tip so that it was aligned with a different, unworn port.

A problem with using such a replaceable or re-positionable sleeve or catheter is that it is not always easy to detect when wear has occurred. Conventionally, this problem has not been considered to be a serious because it was assumed that the main effect of wear or debris accumulation was to make it difficult to focus the laser beam on a target. As explained above, however, the problem is much more serious than heretofore appreciated, because of the coagulation or sloughing of the targeted tissue that can result from firing the laser though a worn or obstructed aperture or window in a sleeve or catheter.

One way to prevent treatment side-effects caused by tissue coagulation or sloughing would be to replace the sleeve or catheter after every use or after a fixed period of time. However, this method does not account for wear that might occur during an extended treatment procedure, and is wasteful from a cost or time standpoint. On the other hand, a more “seat of the pants” approach, in which a physician decides to replace or re-position a sleeve or catheter only when the physician notices an increase in treatment times, can easily result in the occurrence of coagulation or sloughing before the clinician realizes that the sleeve or catheter is excessively worn.

### SUMMARY OF THE INVENTION

It is accordingly an objective of the invention to provide a method of utilizing a side-firing laser and catheter that prevents coagulation or sloughing of a targeted tissue by establishing a fixed limit, based on objective criteria, on permissible usage of the fiber sleeve or catheter before replacement or re-positioning is required.

The method of the invention is not limited to the specific replaceable or multiple-port sleeves or catheters disclosed in the above-cited copending patents and publications, but rather may be used with any replaceable sleeve structure or cap, or any sleeve structure or cap with multiple ports through which the laser may be selectively fired.

The method of the invention involves setting a predetermined usage limit based on actual indications of usage of the catheter, or particular port in the case of a multiple-port sleeve, and replacing the catheter or switching to a different port when the predetermined usage limit is exceeded. In a preferred embodiment of the invention, the usage limit is based on laser energy delivered to the fiber. Since wear is a function of the accumulated amount of laser energy delivered through the sleeve or catheter, this provides an objective indication of when the sleeve or catheter should be replaced or re-positioned.

Further, because different lasers have different energy outputs, the method of replacing or re-positioning a sleeve or catheter based on the amount of laser energy delivered can be used with all types of lasers, including both older high energy lasers and the relatively low energy green lasers currently used in ablation procedures.

### BRIEF DESCRIPTION OF THE DRAWINGS

FIG. 1 is a flowchart of the method of a preferred embodiment of the current invention.

FIGS. 2 and 3 are isometric views showing sleeves or catheters to which the method of FIG. 1 may be applied.

FIG. 4 includes cross-sectional side views of a side-firing laser system for use with the sleeves or catheters of FIGS. 2 and 3.

### DETAILED DESCRIPTION OF THE PREFERRED EMBODIMENTS

Throughout the following description and drawings, like reference numbers/characters refer to like elements. It should be understood that, although specific exemplary embodiments are discussed herein there is no intent to limit the scope of present invention to such embodiments. To the contrary, it should be understood that the exemplary embodiments discussed herein are for illustrative purposes, and that modified and alternative embodiments may be implemented without departing from the scope of the present invention.

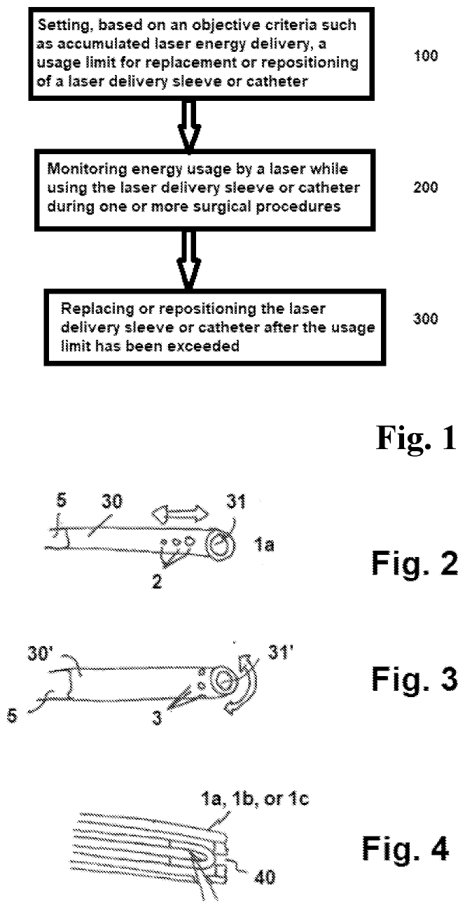

As shown in FIG. 1, the method of the invention includes a step 100 of setting a predetermined usage limit on the catheter based on an objective criteria related to wear of the sleeve or catheter. An example of such an objective criterion is the accumulated energy output of the laser. The more laser energy that is delivered through the sleeve, the greater the wear or degradation. For example, the usage limit might be set at 14000 Joules.

In step 200, after the usage limit is set, the objective criterion is monitored each time a sleeve or catheter is used in a procedure, for example, by recording energy output of the laser. One way to monitor the energy output of the laser would be multiply the energy output per unit time by the total time that laser energy is output. The laser may be equipped with circuitry and/or software to carry out the monitoring and sound an alarm or provide an indication when the usage limit is exceeded, or the energy output may be recorded manually based on treatment times. In either case, when the usage limit is exceeded, the laser delivery sleeve or catheter is replaced or re-positioned. It is likely that replacement or re-positioning will not be required at the exact moment that the usage limit is exceeded, but rather at a convenient break in the treatment procedure, after completion of a current treatment procedure, or at the end of the day that the usage limit is exceeded.

While the method of the invention may be used in connection with sleeves or catheters that are designed to be replaced when worn, it is also possible to use the inventive method with sleeves or catheters that can be re-positioned by moving a worn section of the sleeve or catheter out of the path of the laser. Such re-positionable sleeves or catheters are illustrated in FIGS. 2-4.

FIGS. 2 and 3 show sleeves or catheters 1a and 1b, each having a cylindrical main body 30,30′ made of ceramic, glass, ETFE, PTFE, etc. that may be coupled or affixed to or integral with an introducer 5 through which the laser delivery fiber or an optical fiber assembly is inserted. The main bodies 30,30′ include respective openings or apertures 2, 3 that may generally correspond to a radially-extending lasing opening or aperture described in the above-cited U.S. patent application Ser. No. 15/957,085, filed Apr. 19, 2018, which has a size that is too small for a substantial amount of a liquid such as irrigating fluid to pass, but which is just large enough to permit passage of most or all of the laser beam. Such an opening or aperture has the advantage that any liquid present in the path of the beam will be vaporized or turned to steam, forming an air channel from the fiber to the tissue in order to minimize absorption of the laser beam, and therefore optimizing the amount of energy delivered to the tissue for therapeutic purposes.

Since the size of the opening depends on the cross-sectional dimensions of the beam, i.e., on the beam width, diameter, or Gaussian beam radius (1/e² value), a single opening can only be used with a beam having particular dimensions. Over time, wear will cause the dimensions of the first opening to change, at which time the sleeve can be re-positioned to another opening having the same non-worn dimensions as the first opening.

As shown in FIGS. 2 and 3, the openings 2, 3 can be arranged linearly or circumferentially around the catheter, and may include apertures or openings of different sizes to accommodate different lasers, as well as multiple apertures or openings of each size that enable the laser to re-positioned from a worn opening to a new opening of the same size, by either linearly moving the lasing tip of the optical fiber and/or by rotating the tip, depending on the arrangement of the openings. For example, re-positioning of the sleeve or catheter may involve moving the sleeve or catheter, from a position in which one of the openings is in a path of the laser beam that extends from an angled surface in the fiber to a targeted tissue, to a position in which another of the openings is in the path of the laser beam.

Optionally, one or more of the openings in the illustrated sleeves or catheters may be provided with a diffuser or lens (not shown). In addition, each of the openings may be formed as a knock out, pre-drilled, or pre-scored section that forms a temporary plug and pops out when exposed to a laser beam. When one opening is used and excessively widened or drilled out by passage of the laser, the laser can be moved to the location of another knock out, which pops out to form an opening of a desired size so that the surgical procedure can continue without undue interruption.

Although the term “catheter” is used herein, it will be appreciated that the catheter may be referred to as an introducer or sleeve, that it is a cylindrical or generally cylindrical structure, and that the catheter may be inserted into the patient directly or via a laser endoscope, cystoscope, or resectoscope. In addition, it will be appreciated by those skilled in the art that the optical fiber may be included in an optical fiber assembly or be provided with an additional cap or ferrule through which the laser is fired. Such a cap or ferrule may further be wholly or partly made of a laser-transparent material, or include an opening to permit passage of the laser.

What is claimed is:

1. A method of preventing coagulation or sloughing of a tissue caused by firing of a laser beam through a worn sleeve or catheter, or by debris accumulation on the sleeve or catheter, comprising the steps of:
setting, based on an objective criterion, a usage limit for replacement or re-positioning of the sleeve or catheter;
monitoring the objective criterion during one or more surgical procedures in which the laser beam is fired through the sleeve or catheter; and
replacing or re-positioning the sleeve or catheter after the usage limit has been exceeded.

2. A method as claimed in claim 1, wherein the objective criterion is an accumulated energy delivered through the sleeve or catheter during the one or more surgical procedures.

3. A method as claimed in claim 2, wherein the step of setting the usage limit is a treatment time divided by an energy output of the laser, in units of energy per unit of time, and
wherein the step of monitoring the objective criteria comprises a step of monitoring accumulated usage time of the laser.

4. A method as claimed in claim 1, wherein the sleeve or catheter is replaceable.

5. A method as claimed in claim 1, wherein the sleeve or catheter is re-positionable relative to a tip of the fiber.

6. A method as claimed in claim 5, wherein the sleeve or catheter is linearly or rotatably movable relative to the fiber.

7. A method as claimed in claim 1, wherein the sleeve or catheter includes a plurality of openings, and wherein re-positioning of the sleeve or catheter involves moving the sleeve or catheter, from a position in which one of the openings is in a path of the laser beam from an angled surface of the fiber to a targeted tissue, to a position in which another of the openings is in a path of the laser beam.

8. A method as claimed in claim 1, wherein the step of monitoring energy usage is carried out by monitoring software or circuitry included in the laser to measure the accumulated energy output of the laser overtime.

9. A method as claimed in claim 8, wherein the monitoring software or circuit activates an alarm when the usage limited is exceeded.
